(19) United States
(12) Patent Application Publication
(10) Pub. No.: US 2008/0206866 A1
(43) Pub. Date: Aug. 28, 2008

(54) Reagency and Method for Preventing Time-Dependent Expression in Biological Cells

(75) Inventors: Veit Zieglschmid, Niedersachsen (DE); Oliver Bocher, Zurich (CH); Winfried Albert, Penzberg (DE)

Correspondence Address: FULBRIGHT & JAWORSKI L.L.P., 600 CONGRESS AVE., SUITE 2400, AUSTIN, TX, 78701, US

(73) Assignee: ANDNAGEN AG, HANNOVER-LANGENHAGEN (DE)

(21) Appl. No.: 11/909,142

(22) PCT Filed: Mar. 22, 2006
(86) PCT No.: PCT/EP2006/002642

§ 371 Date: Apr. 25, 2008

(30) Foreign Application Priority Data
Mar. 22, 2005 (DE) 10 2005 013 261.8

(51) Int. Cl. C12N 5/06

(52) U.S. Cl. 435/375

(57) ABSTRACT

The present invention relates to a reagent for prevention of time-dependent, induced expression in biological cells in a sample. It likewise relates to a method for this purpose and also to cell preparations treated in this manner, the reagent for prevention of time-dependence induced expression in biological cells in a sample ex vivo containing a formaldehyde donor.

The present invention relates to a reagent for prevention of time-dependent, induced expression in biological cells in a sample. It relates likewise to a method for this purpose and also to cell preparations treated in this manner.

Reagents of this type and methods of this type are required in particular in the field of molecular-biological analysis and medical diagnostics, in particular tumour diagnostics.

Many detection methods aim to analyse gene expression at mRNA level. Expressed genes can thereby be evaluated as an indication of special cells. However the expression pattern of all tested genes can also be used to draw conclusions about the state of a cell or, by comparing two biological samples, to characterise the differences thereof.

It has been described that, between the taking of the sample and the analysis of the sample, the result can be decomposition of transcribed mRNA in the sample as a result of changes in the ambient conditions of the sample subsequent to removal of the sample (Becker S. et al., 2004 Clin Chem 50 (4), 785-786).

Surprisingly however, a different serious problem occurs in the case of the methods described in the application for detecting cell-associated mRNA and in fact a time-dependent, illegitimate expression of mRNA ex vivo (ex vivo=outwith the human or animal body). After taking the sample, the expression of new mRNA can be induced. This time-dependence-induced expression falsifies the analysis result. The present invention is engaged with this problem.

An illegitimate time-dependence-induced expression of this type is described already in a large number of publications, for example in Baechler et al., Genes Immun. Vol. 5, p. 347 to 353 (2004).

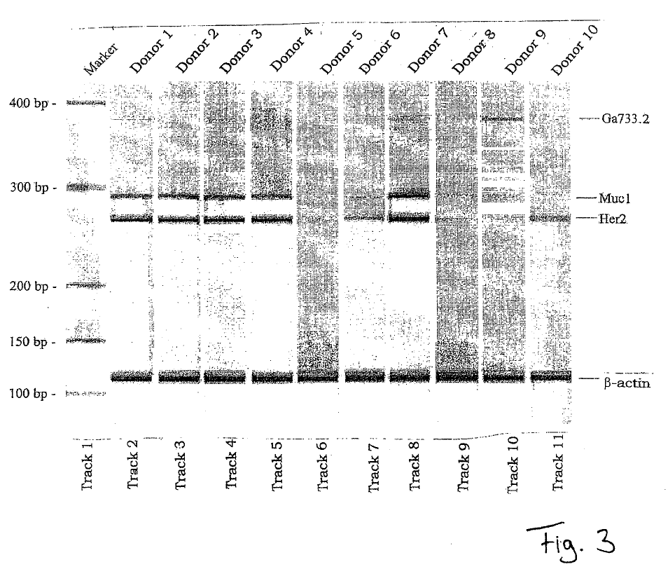

In recent times, highly sensitive analysis methods have been developed, for example as in EP 02 732 726 A1. The detection of tumour cells in human blood according to the method of EP 02 732 726 is dependent upon the fact that the expression status at the time of taking blood is maintained up to analysis. With the method described in EP 02 732 726, tumour cells are enriched immunomagnetically via at least two antibodies, the mRNA of which is isolated and the expression of at least two-tumour associated mRNA markers is examined. Even after a few hours (24 h value shown in FIG. 3), transcripts are detected however without special treatment of the blood samples which were not present at the time of taking blood. This illegitimate transcription leads to a false-positive result.

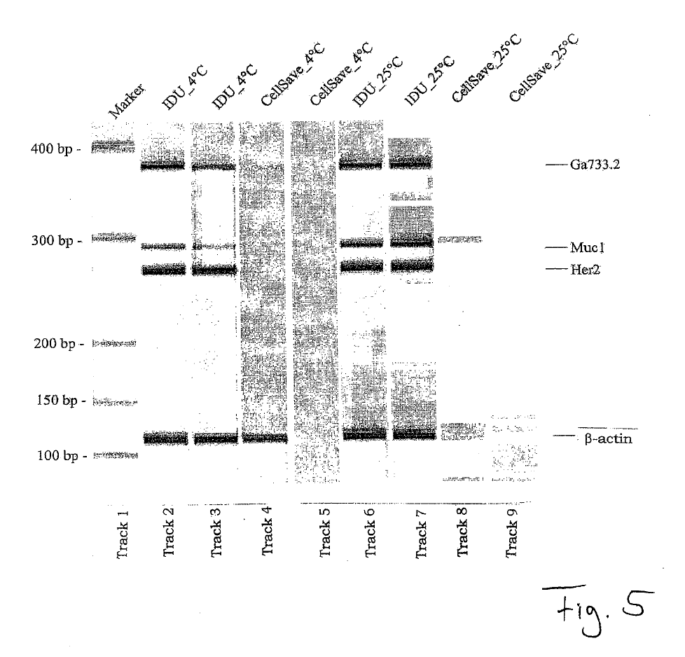

Described methods which aim merely to prevent the decomposition of any mRNA present and also to stabilise the morphology and antigen structure of cells are not suitable for combination with the method of EP 02 732 726. For example there were tested the reagents CellSave® (Immunicon Corporation, Huntingdon Valley, Pa., USA) and Cyto-Chex® (Streck Laboratories, Omaha, Nebr., USA), with which samples were stabilised against decomposition of mRNA according to the manufacturer's instructions. However it was thereby shown that detection of tumour cells according to the method EP 02 732 726 is no longer possible therewith since no meaningful results can be achieved (see FIG. 5). The problem of time-dependent induced expression is accordingly not resolved with the known methods.

The present invention therefore aims to make available a reagent and also a method for prevention and reduction of time-dependent induced expression in biological cells in a sample and the treated cell preparations thereof.

This object is achieved by the reagent according to claim 1, the cell preparation according to claim 9 and also the method according to claim 15. Advantageous developments of the reagent, of the cell preparation and of the method for preventing time-dependent induced expression are given in the respective dependent claims.

The present invention therefore deals successfully for the first time with preventing mRNA new synthesis (time-dependent, induced expression). When using highly sensitive, modern analysis methods, this has at least just as much importance as the previously considered stabilisation of the surface antigens or the mRNA of the target cells. Only in this way is it possible to characterise unequivocally the target cells by means of their mRNA expression.

It was found surprisingly that the addition of a formaldehyde donor to a biological sample in a very small concentration, namely in a concentration ≦0.075% (w/v), advantageously <0.045% (w/v), advantageously ≦0.025% (w/v), leads to prevention of the time-dependent, illegitimate expression. There can be used as formaldehyde donor thereby imidazolidinyl urea (IDU), diazolidinyl urea (DU), dimethylol-5,5-dimethylhydantoin, dimethylol urea, 2-bromo-2-nitropropane-1,3-diol (bronopol), 1,3-bis(hydroxymethyl)-5,5-dimethylimidazolidine-2,4-dione (DMDM-hydantoin), N5-methyl-tetrahydrofolic acid, N10-methyl-tetrahydrofolic acid or a mixture thereof.

With the reagent according to the invention and also with the method according to the invention, it is therefore possible for the first time to prevent the time-dependent induced expression in biological cells in a sample ex vivo and also to enable preservation of disseminated tumour cells in blood samples of cancer patients.

For this purpose, after blood removal, the reagent, if necessary dissolved in phosphate-buffered common salt solution (PBS) and also with the addition of anticoagulant EDTA in the indicated final concentration, is mixed immediately with the blood. The sample can then be stored over a period of several days, for example up to at least 72 h, at 4° C. without illegitimate expression occurring within the sample. In addition, further cell-stabilising substances, such as e.g. polyethylene glycol (PEG) and/or protease inhibitors, can be added to the sample.

In order to demonstrate the effect according to the invention, the effect of the supplements according to the invention was examined in the following examples with respect to the specificity of tumour cell detection by means of analysis of blood samples of healthy donors and also of tumour patients. In addition, the effect of the supplements according to the invention was examined with respect to the sensitivity of the tumour cell detection by means of analysis of blood samples of healthy donors with inoculated tumour cells. The analysis is thereby effected by means of preceding cell selection and subsequent expression profile analysis, as are described in the European Patent application EP 02 732 726. This European Patent application is adopted in its entirety in the present application with respect to the analysis method disclosed there.

Furthermore, the clinical sensitivity was determined by means of analysis of stabilised blood samples of breast and colorectal cancer patients. All the examined patients were at an advanced stage of illness (M1) and were treated with palliative therapy. The supplements according to the invention were mixed with the blood samples immediately after removing the blood samples and were analysed immediately, after 24 h, 48 h and after 72 h storage at 4° C.

It should thereby be mentioned that the following examples relate in fact entirely to blood samples but the method according to the invention is also suitable for testing other cell suspensions or tissue samples. Cell suspensions can thereby be body fluids or also tissue cells in suspension, e.g. tissue pieces, tissue sections and the like.

There are shown

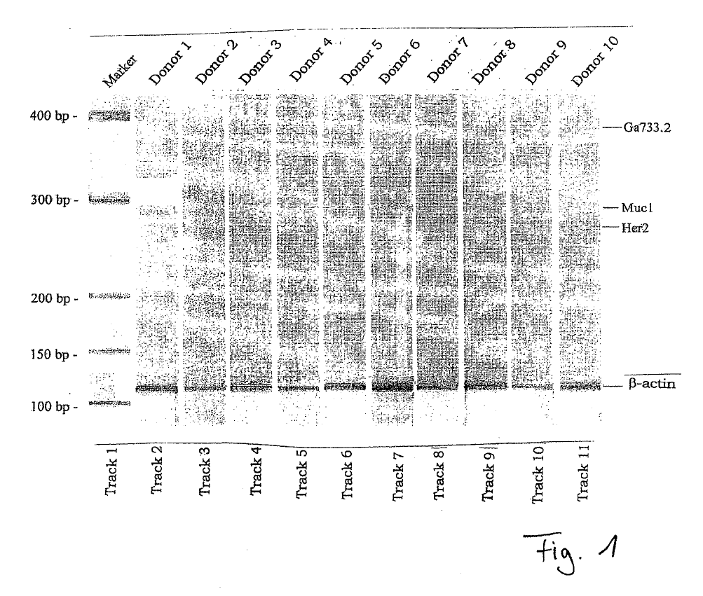

FIG. 1 detection of time-dependent induced transcription in IDU-treated blood samples 24 h after removal;

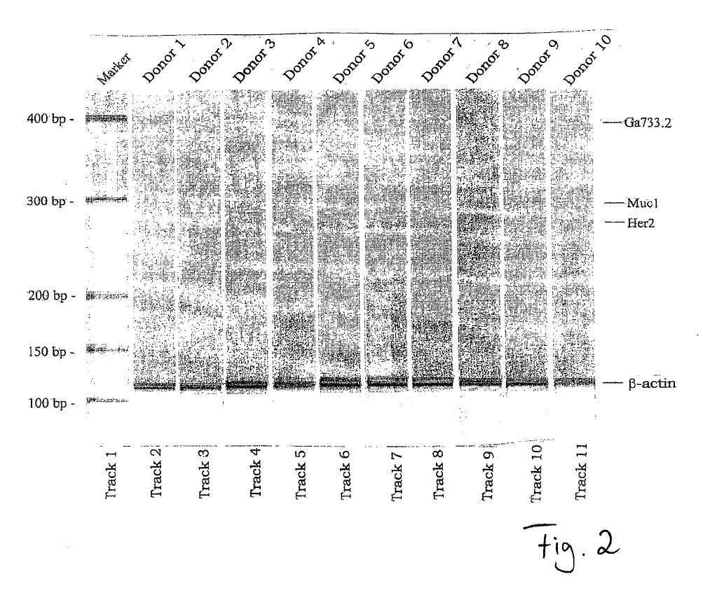

FIG. 2 detection of time-dependent induced transcription in IDU-treated blood samples 48 h after removal;

FIG. 3 detection of time-dependent induced transcription in non-treated blood samples;

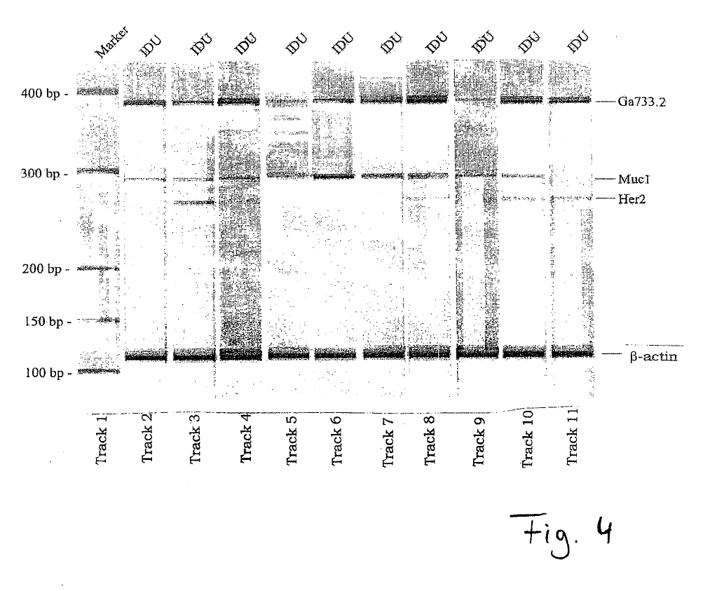

FIG. 4 detection of 2 breast tumour cells in IDU-treated blood samples;

FIG. 5 detection of breast tumour cells in blood samples treated with various systems;

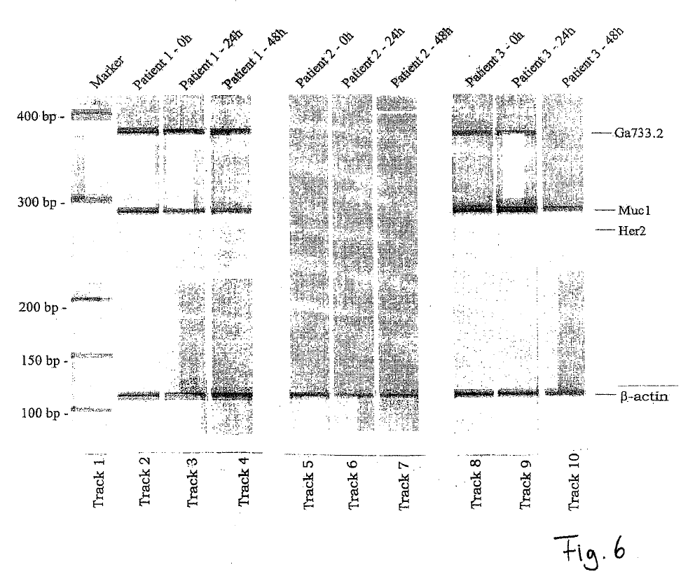

FIG. 6 typical examples of the detection of disseminated tumour cells in IDU-treated blood samples of breast cancer patients;

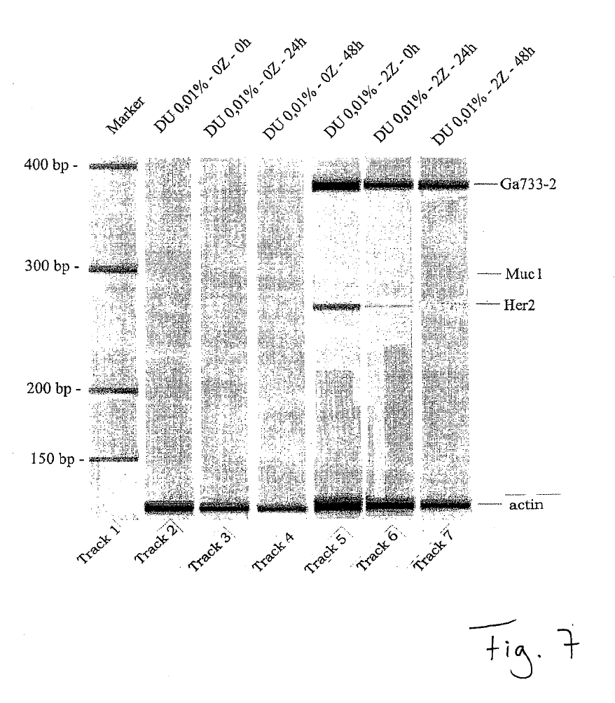

FIG. 7 detection of time-dependent induced transcription in DU-treated blood samples;

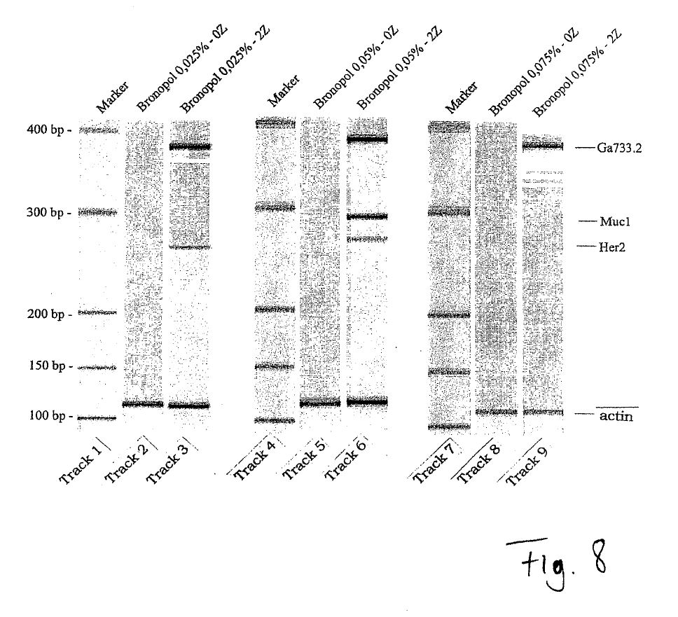

FIG. 8 detection of time-dependent induced transcription in bronopol-treated blood samples.

In all the examples which are represented subsequently, an analysis was implemented as described in EP 02 732 726. Antibodies were thereby coupled to magnetic particles for cell selection. The antibodies represented subsequently in Table 1 were thereby used as antibodies.

TABLE 1

| Antigen | Clone | Company |
|---|---|---|
| epithelial membrane antigen | GP1.4 | Novocastra |
| epithelial antigen | Ber-EP4 | DAKO |
| Muc 1 | HMPV | Pharmingen |

The magnetic particles were thereby used in a concentration of 4×10⁸ beads/ml (CELLection™ Pan Mouse IgG Kit, company Dynal). The ratios between the antibody concentration and the antibodies coupled thereto are reproduced in Table 2.

TABLE 2

| | Antibody | μl antibody/25 μl |
|---|---|---|
| Clone | concentration | particles |
| BerEP4 | 0.32 mg/ml | 4 μl |
| HMPV | 0.5 mg/ml | 4 μl |
| GP1.4 | 0.2 mg/ml | 0.3 μl |

The magnetic particles prepared in this way were mixed in equal parts and added to the blood (EDTA) in 100 μl PBS per 5 ml sample.

After 30 min incubation in an overhead agitator, the magnetic particles which were present possibly as cell-antibody magnetic particle complexes, were washed three times with PBS by means of a magnetic particle concentrator (MPC®-S, company Dynal) and the adhering cells were treated subsequently corresponding to the mRNA isolation protocol described subsequently.

The mRNA isolation was effected by means of Oligo(dT)-coupled magnetic particles, Dynabeads® mRNA Direct™ Micro Kit (company Dynal). This isolation was effected in accordance with the manufacturer's instructions indicated in the kit.

Following the isolation of the mRNA was a reverse transcription in which the mRNA is transcribed into cDNA.

TABLE 3
Components of cDNA synthesis
The cDNA synthesis is effected in a 20 μl reaction batch

| Components | Volumes | |
|---|---|---|
| mRNA | 29.5 | μl |
| 10 × RT buffer | 4 | μl |
| dNTP mix | 4 | μl |
| RNase-inhibitor (RNAsin, Promega) | 0.5 | μl |
| reverse transcriptase | 2 | μl |

The cDNA synthesis (Table 3) was effected at 37° C. for one hour with subsequent inactivation of the reverse transcriptase by heating for 5 minutes at 95° C. and subsequent cooling on ice. For this purpose, a Sensiscript Reverse Transcriptase Kit, company Qiagen, Hilden, was used according to the protocols indicated there.

Subsequent to the transcription of mRNA into cDNA, a polymerase chain reaction (PCR) with β-actin was effected as internal control.

The oligonucleotides cited in Table 4 were thereby used as PCR primer for amplification of cDNA.

TABLE 4
List of PCR primers

| | | |
|---|---|---|
| | | PCR |
| Primer name | Sequence 5′ -> 3′ | product |

Tumour marker

| | | |
|---|---|---|
| GA733.2 sense | AATCGTCAATGCCAGTGTACTTCA | 383 bp |
| GA733.2 sense | TAACGCGTTGTGATCTCCTTCTGA | |
| CA15-3 sense | TCAGCTTCTACTCTGGTGCACAAC | 293 bp |
| CA-15-3 | TGGTAGTAGTCGGTGCTGGGATCT | |
| antisense | | |
| Her-2 sense | CCCAGTGTGTCAACTGCAGCCAGT | 270 bp |
| Her-2 sense | CAGATGGGCATGTAGGAGAGGTCA | |

Internal control

| | | |
|---|---|---|
| Actin sense | CTGGAGAAGAGCTACGAGCTGCCT | 114 bp |
| Actin antisense | ACAGGACTCCATGCCCAGGAAGGA | |

The PCR was implemented with the batch indicated in Table 5.

TABLE 5
PCR batch
The PCR synthesis was effected in a 50 μl reaction batch

| Components | Volumes | |
|---|---|---|
| cDNA | 8 | μl |
| Primer | 4 | μl |
| HotStarTaq | 25 | μl |
| H₂O | 13 | μl |

The PCR conditions (number of cycles, management of cycles etc.) are indicated in Table 6; temperature changes were effected at 2° C./s.

TABLE 6
PCR conditions

| | | | |
|---|---|---|---|
| Preceding denaturation | 95° C. | 15 min | |
| Cycle | | | |
| 1. Denaturation | 94° C. | 1 min | |
| 2. Annealing | 60° C. | 1 min | 35 cycles |
| 3. Extension | 72° C. | 1 min | |
| Final extension | 72° C. | 10 min | |
| | 4° C. | Pause | |

The thus produced amplificates of the cDNA were separated electrophoretically by means of a bioanalyser 2100 (company Agilent). For this purpose, 1 μl of the PCR product was separated in the bioanalyser on a DNA chip 1000 and the separation result was documented electronically. The analysis result is found to be positive if the band intensity at least of one tumour marker is >6.7. The unit “peak level” of the Agilent corresponds to the band intensity. Peaks with a level of <4.0 are evaluated as negative. Peaks, the level of which is 4.0 to 6.7, can be evaluated neither as positive nor as negative. In this way FIGS. 1 to 6 were produced and evaluated.

FIG. 1 shows the detection of time-dependent induced transcription in blood samples treated with IDU.

Track 1 thereby shows a ladder with size markers, tracks 2 to 11 show the amplified DNA fragments of the tumour-associated transcripts which are to be detected in ten different healthy donors and an internal control of β-actin. The position of the respective bands for β-actin and the three tumour-associated transcripts GA733-2 (384 bp), MUC-1 (292 bp) and Her-2 (265 bp) are described in FIG. 1.

The samples of the ten healthy donors were thereby replaced with 0.045% (w/v) IDU and subsequently stored for 24 h at 4° C. The amplified DNA fragments were detected after analysis of the blood samples by means of high voltage capillary gel electrophoresis in a Bioanalyser 2100 (Agilent technologies). It can be detected that the analysis in all ten healthy donors (donor 1 to donor 10 in tracks 2 to 11) has come out negative (peak levels below threshold value). This means that no illegitimate time-dependent induced expression can be detected.

FIG. 2 shows the same test but now after storage of the samples of the ten healthy donors for 48 h. Here also, still no time-dependent expression of tumour-associated transcripts was able to be detected.

FIG. 3 shows, in contrast, the same blood samples as in FIG. 1 and in FIG. 2 but without the addition of 0.025% (w/v) IDU. It can be detected that illegitimate expression of the tumour-associated transcripts occurred with these untreated blood samples.

In the tests according to FIGS. 1 to 3, it was detected unequivocally that the illegitimate time-dependent induced expression in cells within a cell preparation can be suppressed effectively with the indicated concentrations of a formaldehyde donor. The supplements according to the invention obtain high specificity.

FIG. 4 shows, in contrast, the detection of breast tumour cells in blood samples of ten healthy donors which were treated with 0.045% (w/v) IDU (tracks 2 to 10). Two breast cancer cells (MCF7) per 5 ml blood were inoculated into these samples. The represented test on the expression pattern of the tumour-associated transcripts Her-2, MUC-1 GA733.2 was implemented after storage of the blood samples at 4° C. over 48 h. All further analysis parameters correspond to those in FIGS. 1 to 3. It can be detected that the tumour-associated transcripts Her-2, MUC-1 and GA733.2, which were introduced into the blood samples via the inoculation of MCF7 breast cancer cells, were detected unequivocally. FIG. 4 hence shows unequivocally that even such low cell numbers, such as 2 cells/5 ml blood, can still be detected after 48 h if the blood sample has been treated according to the invention, without the detection being impeded by illegitimate time-dependence-induced expression. Loss of sensitivity in the sought tumour cell detection is therefore likewise prevented by the method according to the invention.

FIG. 5 shows comparative tests with the commercially available stabilisation solution “CellSave” by Immunicon. For this purpose, respectively 5 breast cancer cells (HCC1954) were inoculated in 5 ml blood of healthy donors. Two of the samples were treated according to the invention with 0.025% (w/v) IDU and stored at 4° C. (tracks 2 and 3). Two further samples were treated in a corresponding manner but stored at 25° C. (tracks 6 and 7). Two further samples were stabilised with the product CellSave according to the manufacturer's instructions and stored at 4° C. (tracks 4 and 5). The last two samples in FIG. 5 (tracks 8 and 9) were likewise stabilised with CellSave according to the manufacturer's instructions and stored at 25° C.

As can be detected, the expression of the tumour markers Her-2, MUC-1 and GA733.2 in the inoculated breast cancer cells and also of the marker β-actin in the samples treated according to the invention (tracks, 2, 3, 6, 7) is detected. With the samples stabilised with the commercial stabilisation solution CellSave (tracks 4, 5, 8, 9), sensitive detection neither of the marker β-actin nor of the tumour markers Her-2, MUC-1 and GA733.2 was possible.

This shows that, in contrast to prior art, a very effective preservation of the expression pattern in a biological sample is achieved by the present treatment according to the invention which permits the subsequent analysis to be implemented highly selectively and sensitively.

FIG. 6 shows the analysis of blood samples of breast cancer patients directly after removal of the blood samples, after 24 h and also after 48 h. All these samples were mixed immediately after blood removal with 0.025% (w/v) IDU and stored at 4° C.

In tracks 2 to 4, FIG. 6 shows the test results of the blood sample of a patient directly after blood removal, after 24 h and 48 h after the blood removal. It is shown that the expression pattern remains extensively the same over time and consequently the IDU supplement leads to preservation of the expression pattern. It is thereby crucial that also no illegitimate time-dependent induced expression occurs. This is also the case with the sample of the second patient which was tracked over time in tracks 5 to 7. This obviously has no circulating tumour cells in the blood sample. A time-dependent induced expression was likewise not established. Also the sample of patient 3 in tracks 8 to 10 shows that, with the treatment with 0.025% (w/v) IDU, preservation of the expression pattern was able to be achieved over 48 h.

In summary, it could be established that tumour cell detection in blood samples of breast cancer patients which were treated with 0.025% (w/v) IDU immediately after removal, after 24 h and after 48 h shows uniform results, i.e. blood samples in which tumour cells could be detected at the time of removal, also show, after 24 h and 48 h, a positive tumour cell detection, whilst samples without tumour cells also remained negative after 24 and 48 h, i.e. no false-positive results are produced which could be produced by a time-dependent expression ex vivo. The tested blood samples therefore show, over a period of 48 h, a constant expression pattern, i.e. samples which were initially negative remained negative and positive samples remained positive.

FIG. 7 shows the detection of time-dependent induced transcription in blood samples treated with DU. The detection was effected according to the protocols described for IDU.

Track 1 thereby shows a ladder with size markers, tracks 2 to 4 show the amplified DNA fragments of the tumour-associated transcripts to be detected in a healthy donor and of an internal control of β-actin. Tracks 5 to 7 show the transcripts of samples with 2 inoculated cells of the tumour cell line Calu-3 at the times 0 h, 24 h and 48 h. The position of the respective bands for β-actin and the three tumour-associated transcripts GA733-2 (384 bp), MUC-1 (292 bp) and Her-2 (265 bp) are described in FIG. 7.

The samples were thereby mixed with 2 inoculated Calu-3 cells with 0.01% (w/v) DU and subsequently were stored at 4° C. for max. 48 h. The amplified DNA fragments were detected after analysis of the blood samples by means of high voltage capillary gel electrophoresis in a Bioanalyser 2100 (Agilent technologies). It can be detected that the analysis in all the samples without inoculated cells (tracks 2 to 4) was negative (peak levels below threshold value). This means that no illegitimate time-dependent induced expression can be detected.

It can be detected in tracks 5 to 7 that tumour-associated transcripts, which were introduced into the blood samples via inoculation of Calu-3 cancer cells, are detected unequivocally. FIG. 7 hence shows unequivocally that even small cell numbers can still be detected after 48 h if the blood sample has been treated according to the invention without detection being impeded by illegitimate time-dependent induced expression. Loss of sensitivity in the sought tumour cell detection is therefore prevented by the method according to the invention.

FIG. 8 shows the detection of time-dependent induced transcription in blood samples treated with bronopol (2-bromo-2-nitropropane-1,3-diol).

Tracks 1, 4 and 7 thereby show a ladder with size markers, tracks 2, 5 and 8 show the amplified DNA fragments of the tumour-associated transcripts to be detected in a healthy donor and of an internal control of β-actin. Tracks 3, 6 and 9 show the transcripts of samples with 2 inoculated cells of the tumour cell line Calu-3. The position of the respective bands for β-actin and the three tumour-associated transcripts GA733-2 (384 bp), MUC-1 (292 bp) and Her-2 (265 bp) are described in FIG. 8.

The samples of a healthy donor were thereby mixed with 0.025% (tracks 2 and 3), 0.05% (tracks 5 and 6) and 0.075% (w/v) (tracks 8 and 9) of bronopol and subsequently were stored at 4° C. for 24 h. The amplified DNA fragments were detected after analysis of the blood samples by means of high voltage capillary gel electrophoresis in a Bioanalyser 2100 (Agilent technologies). It can be detected that the analysis in all three concentrations of bronopol (tracks 2, 5 and 8) was negative (peak levels below threshold value). This means that no illegitimate time-dependent induced expression can be detected. Tracks 3, 6 and 9 show that tumour-associated transcripts, which were introduced into the blood samples via inoculation of Calu-3 cancer cells, are detected unequivocally. FIG. 8 hence shows unequivocally that even such small cell numbers, such as 2 cells, can still be detected after 24 h if the blood sample has been treated according to the invention without detection being impeded by illegitimate time-dependent induced expression. Loss of sensitivity in the sought tumour cell detection is therefore likewise prevented by the method according to the invention.

The above-represented test results unequivocally show both high effectiveness and reliability of the reagent according to the invention and of the method according to the invention in the treatment of cell preparations, in particular of disseminated tumour cells, in the blood of breast cancer patients. Comparable results were achieved in the case of colorectal cancer patients.

The present test results therefore show that prevention of a time-dependent induced expression in biological cells in a sample ex vivo is achieved. Furthermore, disseminated tumour cells in the peripheral blood of breast and colorectal cancer patients was able to be detected by the addition of the reagents according to the invention. No time-dependent induced transcription was detected over 48 h. In untreated blood samples, a very pronounced time-dependent induced expression was however observed even after 24 h.

The reagents according to the invention and the method according to the invention thus enable prevention of a time-dependent induced expression in biological cells in a sample (in particular ex vivo) and at the same time preservation of disseminated tumour cells in the peripheral blood of cancer patients. They have therefore a high logistical and diagnostic value since fairly long transportation and a fairly large temporal interval between removal of the sample and analysis of the sample is made possible herewith. The reagents according to the invention and the method according to the invention are thus suitable for use in blood removal systems.

1. A method for prevention of time-dependent illegitimate expression of mRNA in a biological cell in a sample ex vivo comprising mixing the sample with a reagent comprising a formaldehyde donor.

2. The method according to claim 1, wherein the resulting mixture comprises the formaldehyde donor in a concentration of ≦0.075% (w/v).

3. The method according to claim 2, wherein the resulting mixture comprises the formaldehyde donor in a concentration of ≦0.045% (w/v), advantageously ≦0.025% (w/v).

4. The according to claim 1, wherein the formaldehyde donor comprises imidazolidinyl urea, diazolidinyl urea, dimethylol-5,5-dimethylhydantoin, dimethylol urea, 2-bromo-2-nitropropane-1,3-diol, 1,3-bis(hydroxymethyl)-5,5-dimethylimidazolidine-2,4-dione (DMDM-hydantoin), N5-methyl-tetrahydrofolic acid, N10-methyl-tetrahydrofolic acid or a mixture thereof.

5. The method according to claim 8, wherein the liquid is a buffer, physiological common salt solution or the like.

6. The method according to claim 1, wherein the biological cell is separated by means of antibodies or via other conventional methods.

7. A method for the detection of a tumor cell comprising preventing time-dependent expression of mRNA in a biological cell within a sample comprising mixing the sample with a reagent comprising a formaldehyde donor.

8. The method of claim 7, wherein in the sample is a body fluid, blood, liquor, ascites, urine, a tissue piece or a suspension which contains cells from tissue sample.

9. The method of claim 7, wherein said reagent further comprises a fluid.

10. The method of claim 1, wherein said reagent further comprises a fluid.


SEQUENCE LISTING (8 sequences)

SEQ ID NO: 1 - DNA, 24 bp, Artificial
  Synthetic primer
aatcgtcaatgccagtgtacttca

SEQ ID NO: 2 - DNA, 23 bp, Artificial
  Synthetic primer
taacgcttgtgatctccttctga

SEQ ID NO: 3 - DNA, 24 bp, Artificial
  Synthetic primer
tcagcttctactctggtgcacaac

SEQ ID NO: 4 - DNA, 24 bp, Artificial
  Synthetic primer
tggtagtagtcggtgctgggatct

SEQ ID NO: 5 - DNA, 24 bp, Artificial
  Synthetic primer
cccagtgtgtcaactgcagccagt

SEQ ID NO: 6 - DNA, 24 bp, Artificial
  Synthetic primer
cagatgggcatgtaggagaggtca

SEQ ID NO: 7 - DNA, 24 bp, Artificial
  Synthetic primer
ctggagaagagctacgagctgcct

SEQ ID NO: 8 - DNA, 24 bp, Artificial
  Synthetic primer
acaggactccatgcccaggaagga